(19) United States
(12) Patent Application Publication
(10) Pub. No.: US 2026/0062621 A1
(43) Pub. Date: Mar. 5, 2026

(54) METHOD FOR PRODUCING RESOURCES FROM SEAWEED

(71) Applicants: NIPPON STEEL Chemical & Material Co., Ltd., Tokyo (JP); National University Corporation Shizuoka University, Shizuoka (JP)

(72) Inventors: Yasuji SHICHIJO, Tokyo (JP); Keita YOSHIDA, Tokyo (JP); Idzumi OKAJIMA, Shizuoka (JP)

(73) Assignees: NIPPON STEEL Chemical & Material Co., Ltd., Tokyo (JP); National University Corporation Shizuoka University, Shizuoka (JP)

(21) Appl. No.: 19/106,627

(22) PCT Filed: Aug. 31, 2023
(86) PCT No.: PCT/JP2023/031816

§ 371 Date: Feb. 25, 2025

(30) Foreign Application Priority Data
Aug. 31, 2022 (JP) 2022-137819
Mar. 31, 2023 (JP) 2023-057659

(51) Int. Cl. C10G 1/02; C12N 1/12

(52) U.S. Cl. CPC C10G 1/02; C12N 1/12

(57) ABSTRACT

A process for producing liquid hydrocarbon and carbide using an organic raw material is developed. Disclosed is a method for industrially producing resources from seaweeds, the method including: a first step of performing a low-temperature hydrothermal treatment at from 120° C. to 250° C. using seaweeds and then performing solid-liquid separation to obtain a first liquid substance and a first solid residue having an O/C ratio of from 0.3 to 0.8; and a second step of performing a high-temperature hydrothermal treatment at from 250° C. to 370° C. using a first aqueous phase obtained from the first liquid substance obtained in the first step and then performing solid-liquid separation to obtain a second liquid substance including a heavy oil-containing liquid substance and a second solid residue.

### TECHNICAL FIELD

The present invention relates to a system for producing industrial resources such as useful industrial products and liquid fuels using biomass. In particular, the present invention relates to a method for producing carbon-containing components such as carbide and liquid hydrocarbon by use of seaweed.

### BACKGROUND ART

Utilization of biomass, which is a natural resource, is an essential technology for sustained economic development. In the known art, attempts have been made to produce industrial products such as carbide and liquid hydrocarbon using wood and grass essence mainly containing cellulose as natural resources.

For example, Patent Document 1 discloses a method for obtaining solid, liquid and gaseous fuels from an organic raw material under high pressure heating, the method including: using sediment or dust containing a microbial, plant, or animal biological material as the organic raw material; blocking air to gradually heat the raw material to a conversion temperature of from 200° C. to 600° C.; guiding generated gas and vapor through an appropriate gas separation device and liquid separation device; and maintaining the conversion temperature until generation of gas and vapor is substantially ceased to separate a solid conversion residue, gas, and liquid.

In addition, algae have attracted attention as the organic raw materials. For example, Patent Document 2 discloses a method for producing a heavy oil substance from microalgae, in which the microalgae is kept at a high temperature and a high pressure in the presence of an alkaline substance and in the presence of an aqueous medium to be liquefied into a heavy oil substance.

Patent Document 3 discloses a method for hydrothermally processing biomass, the method including: introducing a biomass feed having a water to biomass ratio of at least 1:1 into a reaction zone, the biomass feed having a phosphorus content; hydrothermally treating the biomass feed under effective hydrothermal treatment conditions to produce a multi-phase product, the multi-phase product including a solids portion containing at least about 80% of the phosphorus content of the biomass feed; and separating the multi-phase product to produce at least a gas phase portion, a liquid hydrocarbon product, and the solids portion.

### CITATION LIST

### Patent Document

Patent Document 1: JP S57-111380 A (Claims, Examples, etc.)

Patent Document 2: JP H06-41545 A (Claims, Examples, etc.)

Patent Document 3: JP 5694559 B (Claims, Examples, etc.)

### SUMMARY OF INVENTION

### Technical Problem

When biomass is industrially used as an organic raw material, productivity is required. The present inventors have focused on large seaweeds typified by kelp seaweed as an organic raw material, and aimed to develop a process for producing liquid hydrocarbon and carbide using the large seaweed.

As a result of detailed studies by the present inventors, it has been found that when a hydrothermal treatment step is performed using seaweeds at a lower temperature, a solid residue containing an organic matter is mainly obtained, and when the hydrothermal treatment step is performed at a higher temperature, a liquid hydrocarbon is mainly obtained, however, a total yield from a raw material biomass is low as a whole, which is industrially disadvantageous.

The problem to be solved by the present invention is to provide a process for producing light oil and heavy oil with high yield using seaweeds.

### Solution to Problem

The present inventors have conducted detailed studies, and resultantly found that when heat treatment is performed using seaweeds, a heavy oil-containing liquid substance can be efficiently obtained by performing heating in two stages of a low-temperature hydrothermal treatment at from 120° C. to 250° C. and a high-temperature hydrothermal treatment at from 250° C. to 370° C., leading to the present invention.

The present invention relates to a method for industrially producing resources from seaweeds, the method including: 3 first step of performing a low-temperature hydrothermal treatment at from 120° C. to 250° C. using seaweeds and then performing solid-liquid separation to obtain a first liquid substance and a first solid residue having an O/C ratio of from 0.3 to 0.8; and a second step of performing a high-temperature hydrothermal treatment at from 250° C. to 370° C. using a first aqueous phase obtained from the first liquid substance obtained in the first step and then performing solid-liquid separation to obtain a second liquid substance including a heavy oil-containing liquid substance and a second solid residue.

Another aspect of the present invention is a method for industrially producing resources from seaweeds, the method further including, in addition to the first step and the second step, a third step of performing a high heat treatment at from 400° C. to 1000° C. using the first solid residue and/or the second solid residue to co-produce carbide at a high yield in addition to a heavy oil-containing liquid substance rich in heavy oil content.

The present inventors have further continued the study, and have conceived to reuse an aqueous phase containing a water-soluble organic matter to be generated in a high-temperature hydrothermal treatment when performing a heat treatment using seaweeds in two stages of a low-temperature hydrothermal treatment at from 120° C. to 250° C. and the high-temperature hydrothermal treatment at from 250° C. to 370° C.

Still another aspect of the present invention is a method for industrially producing resources from seaweeds, the method including: a first step of performing a low-temperature hydrothermal treatment at from 120° C. to 250° C. using seaweeds and then separating and obtaining a first oil phase, a first aqueous phase, and a first solid residue from the treated product; and a second step of performing a high-temperature hydrothermal treatment at from 250° C. to 370° C. using the first aqueous phase obtained from a first liquid substance obtained in the first step, and then performing solid-liquid separation to obtain a second liquid substance including a heavy oil-containing liquid substance and a second solid residue, wherein at least a part of a second aqueous phase obtained from the second liquid substance obtained in the second step is circulated to a step of performing the high-temperature hydrothermal treatment together with the first aqueous phase obtained from the first liquid substance obtained in the first step.

### Advantageous Effects of Invention

According to the present invention, there is provided a process for producing useful liquid hydrocarbon and carbide with high yield using seaweeds. The obtained liquid hydrocarbon can be suitably used for a raw material of carbon black, tar, pitch, fuel oil of various combustion furnaces, and the like. The obtained carbide can be suitably used for activated carbon and the like.

### BRIEF DESCRIPTION OF DRAWINGS

FIG. 1 is a conceptual diagram illustrating an embodiment of the present invention.

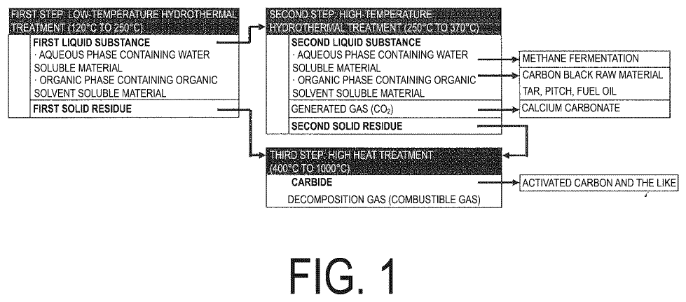

FIG. 2 is a conceptual diagram illustrating another embodiment of the present invention.

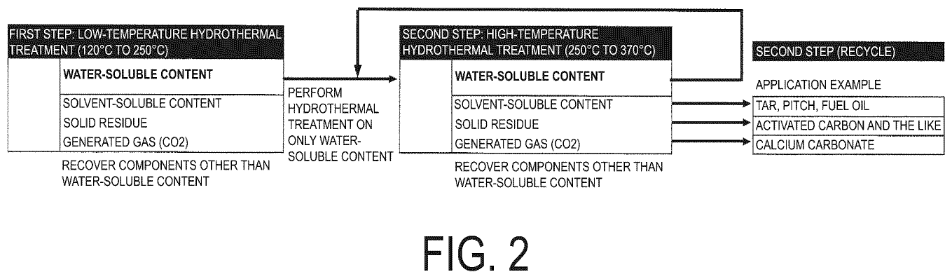

### DESCRIPTION OF EMBODIMENTS

In the present invention, seaweeds are used as a raw material. As the seaweeds, in particular, large seaweeds can be preferably used. Specifically, green algae, brown algae, and red algae are more preferably used. Most preferably, large brown algae typified by kelp seaweed are used.

The reason is that the large seaweeds can maintain a growth rate even if an algal body density is increased as compared with microalgae, and thus are excellent in productivity.

The seaweeds may be naturally grown or may be grown in a culture environment. Seaweeds once dried can also be used. Use of seaweeds grown by performing photosynthesis by sunlight indirectly leads to resource production using natural energy. The seaweeds are crushed and subjected to a first step preferably in a slurry state with a solvent such as water added thereto.

The first step includes a step of performing a low-temperature hydrothermal treatment at from 120° C. to 250° C. using seaweeds, and then a step of separating and obtaining a first oil phase, a first aqueous phase, and a first solid residue from the treated product.

The low-temperature hydrothermal treatment is performed by heating seaweeds in a treatment vessel to a temperature of from 120° C. to 250° C., preferably from 140° C. to 230° C., and more preferably from 160° C. to 210° C. When the treatment temperature in the low-temperature hydrothermal treatment step is lower than 120° C., it is not preferable because a hydrolysis reaction does not proceed. When the temperature is higher than 250° C., decomposition of an organic matter in the solid residue proceeds excessively, which is not preferable.

An amount of water added to the seaweeds is preferably from 1 to 20 times, more preferably from 2 to 15 times the weight of the dried seaweeds. The atmosphere in the treatment vessel is preferably an inert gas, The pressure depends on the treatment temperature, and it is preferable to operate at equal to or more than a saturated vapor pressure at the treatment temperature.

The treatment method may be a batch type or a flow type as long as the treatment conditions can be secured. The treatment vessel to be used is preferably a pressure vessel equipped with a heating device and a mixer.

The treatment time is preferably 3 minutes to 120 minutes, and more preferably 5 minutes to 60 minutes in either case of the batch type or the flow type.

A product obtained by the low temperature hydrothermal treatment is mainly a mixture of a liquid substance and a solid substance. In the present invention, a liquid substance produced by the low-temperature hydrothermal treatment is referred to as a first liquid substance, and a solid substance produced by the low-temperature hydrothermal treatment is referred to as a first solid residue. In addition, a trace amount of combustible gas may be generated.

The treated product obtained by the low temperature hydrothermal treatment is subjected to solid-liquid separation by usual solid-liquid separation operations such as decantation, filtration, and centrifugation to be separated into the first liquid substance and the first solid residue.

The first solid residue is a solid substance having an O/C ratio of from 0.3 to 0.8, preferably from 0.35 to 0.6. Here, the O/C ratio is an amount ratio between the number of oxygen atoms and the number of carbon atoms contained in the solid residue.

When the O/C ratio is less than 0.3, the decomposition of the organic matter proceeds excessively, and a yield of carbide decreases, which is not preferable. On the other hand, when the O/C ratio is more than 0.8, the decomposition of the organic matter does not proceed, and the yield of liquid substance decreases, which is not preferable.

The first solid residue is a precursor of carbide, and serves as a raw material for producing carbide by being subjected to a third step described later. The first solid residue is in a semi-carbonized state as a partially carbonized state. Since the dehydration reaction and the decarboxylation reaction proceed by subjecting the seaweed as the raw material to the hydrothermal treatment, the O/C ratio of the first solid residue is lower than that of the raw seaweed.

While the first liquid substance comprises an aqueous phase containing a water soluble material and an organic phase containing an organic solvent soluble material, the entirety of the first liquid substance or only the aqueous phase as the first aqueous phase separated by an oil-water separation operation is sent to a second step. The first liquid substance may be subjected to the oil-water separation operation to obtain the organic phase containing the organic solvent soluble material separated from the aqueous phase containing the water soluble material, and the organic phase may be combined with a heavy oil-containing liquid substance described later.

The second step includes a step of performing a high-temperature hydrothermal treatment at from 250° C. to 370° C., and then a step of separating and obtaining a second oil phase, a second aqueous phase, and a second solid residue from the treated product.

The high-temperature hydrothermal treatment is performed by heating the first aqueous phase in a treatment vessel to a temperature of from 250° C. to 370° C., preferably from 270° C. to 360° C., and more preferably from 300° C. to 350° C.

When the treatment temperature in the high-temperature hydrothermal treatment step is lower than 250° C., the dehydration reaction hardly proceeds, so that the yield of heavy oil is reduced, which is not preferable. On the other hand, when the temperature is higher than 370° C., the decomposition of the organic matter proceeds excessively, and the yield of liquid substance decreases, which is not preferable.

The atmosphere in the treatment vessel is preferably an inert gas. The pressure depends on the treatment temperature, and is preferably in a pressurized state in a range of from 2.5 MPa to 22 MPa.

The treatment method may be a batch type or a flow type as long as the treatment conditions can be secured, and the treatment vessel to be used is preferably a pressure vessel including a heating device and a mixer. The treatment time is preferably 3 minutes to 60 minutes, and more preferably 5 minutes to 30 minutes in both cases of the batch type and the flow type.

A treated product obtained by the high-temperature hydrothermal treatment is mainly a mixture of a liquid substance and a solid substance. In the present invention, liquid substance produced by the high-temperature hydrothermal treatment is referred to as a second liquid substance, and a solid substance produced by the high-temperature hydrothermal treatment is referred to as a second solid residue. In addition, a decomposition gas is generated, most of the components thereof are CO₂, and can be recovered as calcium carbonate by being trapped with calcium hydroxide.

From the treated product obtained by the high-temperature hydrothermal treatment, the second liquid substance and the second solid residue are obtained by usual solid-liquid separation operations such as decantation, filtration, and centrifugation.

The second solid residue is a precursor of carbide, and serves as a raw material for producing carbide by being subjected to the third step described later. The second solid residue is in a semi-carbonized state as a partially carbonized state. Since the dehydration reaction and the decarboxylation reaction proceed by subjecting the seaweed as the raw material to the hydrothermal treatment, the O/C ratio of the first solid residue is usually lower than that of the raw seaweed.

The second liquid substance includes a second aqueous phase containing a water-soluble organic matter and a second oil phase containing an organic solvent soluble material. The second liquid substance is separated by an oil separation operation to obtain the second aqueous phase and the second oil phase.

The second oil phase obtained in the second step is purified and separated into useful carbon-containing components such as light oil, heavy oil, pitch, and tar by a purification step such as distillation, and can be used as chemical products, fuels, or other industrial products. The first oil phase separated by the oil-water separation operation in the first step may be combined as a raw material in the purification step.

In the present invention, preferably, at least a part of the second aqueous phase obtained in the second step can be circulated in the step of performing the high-temperature hydrothermal treatment in the second step, and subjected to the step of performing the high-temperature hydrothermal treatment together with the first aqueous phase obtained from the first liquid substance obtained in the first step. When the second aqueous phase obtained in the second step is circulated, only water may be removed and concentrated for use. As a method for removing water from the second aqueous phase, the means such as distillation and membrane separation can be used.

As a result, the water-soluble organic matter contained in the second aqueous phase is subjected to the high-temperature hydrothermal treatment again, and an acquired amount of the carbon-containing component increases. A percentage of an amount of the second aqueous phase obtained in the second step to be circulated in the step of performing the high-temperature hydrothermal treatment can be determined by comparing the acquired amount of the carbon-containing Component with energy cost of performing the high-temperature hydrothermal treatment and the like; however, it is preferable to minimize the aqueous phase finally discharged to the outside of the system from the present process.

The main component of seaweeds is polysaccharides. By supplying the seaweeds as the raw material to the first step, the polysaccharide is hydrolyzed to produce a water soluble mixture mainly composed of a Water-soluble low molecular weight compound and/or oligomer. Examples of the organic solvent soluble material include polyvalent unsaturated fatty acids. When the first liquid substance obtained through the first step is supplied to the second step, a considerable amount of the water-soluble organic matter in the first liquid substance becomes heavier and becomes the organic solvent soluble material. At least a part of the organic solvent soluble material in the first liquid substance further becomes heavy, and the yield of carbide increases.

Here, it is essential to combine the first step and the second step in order to advance an industrial resource recovery business of seaweeds with high productivity. When the first step is not performed, the decomposition proceeds excessively in the high-temperature hydrothermal treatment, so that the amount of organic matter in the solid residue is small, and as a result, the yield of carbide is reduced. When the second step is not performed, the decomposition of seaweed does not sufficiently proceed, and the subsequent heaviness hardly occurs, so that a heavy oil content is poor, and as a result, it is not possible to produce the organic solvent soluble material with high yield.

A part of the second aqueous phase obtained in the second step that has not been circulated is preferably subjected to a fermentation step. In the fermentation step, the water-soluble organic matter contained in the second aqueous phase can be fermented by microorganisms to obtain a combustible gas containing methane. The obtained combustible gas can be used as a fuel gas. Carbon dioxide by-produced in the fermentation step can react with lime to produce calcium carbonate.

By supplying the solid residue obtained in the first step and/or the second step to the third step described later, carbide can be co-produced. A preferred embodiment of the present invention includes a third step of performing a high heat treatment using the first solid residue obtained in the first step and/or the second solid residue obtained in the second step to obtain carbide.

The high heat treatment in the third step can be performed according to the use. The high heat treatment is usually performed by heating in an oxygen-free state at a temperature in a range of from 400° C. to 1000° C.

When the temperature of the high heat treatment in the third step is in the range of from 400° C. to 1000° C., the decomposition of the organic matter proceeds, an amount of residual element is small, the quality of carbide is excellent, in addition, it is not necessary to perform the treatment at an unnecessary high temperature, and energy efficiency is good, which is preferable. During the high heat treatment, the atmosphere in the treatment vessel is preferably an inert gas. The pressure is preferably in a range of from a normal pressure to 1 MPa.

The time for the high heat treatment is preferably from 20 minutes to 120 minutes, and more preferably from 30 minutes to 90 minutes. The treatment method may be a batch type or a flow type as long as the treatment conditions can be secured, and the treatment vessel to be used is preferably an incinerator or an electric furnace.

The O/C ratio of the carbide obtained in the third step is preferably from 0 to 0.2, and more preferably from 0 to 0.1. The physical properties of the carbide can be controlled by controlling the temperature and time of the high heat treatment step. The obtained carbide can be suitably used as a raw material of carbide such as activated carbon. According to the present invention described above, a carbon-containing component can be obtained in an industrially usable form from an organic component contained in raw seaweeds.

### EXAMPLES

In the following Examples and Comparative Examples, each component produced was measured by JIS M-8819 Coal and coke-Mechanical methods for element analysis and JIS M-8813 Coal and coke-Determination of elements.

### Example 1

### (First Step)

12 g of dried kelp seaweed (Japanese kelp) was pulverized, mixed with water at a weight ratio of kelp seaweed/water=¼, and subjected to hydrothermal treatment at 180° C. for 60 minutes in a 75 ml SUS pressure-resistant container (autoclave). At this time, the pressure in the autoclave was controlled to 3 MPa or less.

A reaction mixture after the hydrothermal treatment was washed with THE and water, and filtered, and the solid content was separated by filtration and dried to obtain a solid residue (first solid residue). The filtrate was separated into oil and water, and THE was removed from the oil phase to obtain a solvent-soluble content. The aqueous phase was used as it was as a water-soluble content. The solvent-soluble content and the water-soluble content were both referred to a first liquid substance.

In a production ratio of each component in the first step, the gas component was 0%, the water-soluble content of the first liquid substance was 40%, the solvent-soluble content of the first liquid substance was 5%, and the first solid residue was 55% in terms of carbon yield where kelp seaweed as the raw material was 100%. The O/C ratio and the H/C ratio of the first solid residue were 0.46 and 1.27, respectively. The O/C ratio and the H/C ratio of the solvent-soluble content of the first liquid substance were 0.14 and 1.69, respectively.

### (Second Step)

The water-soluble content (0.15 g as carbon) obtained in the first step was heat-treated at 350° C. for 5 minutes in a 10 mL SUS pressure-resistant container (autoclave). At this time, the pressure in the autoclave was controlled to 20 MPa or less.

A reaction mixture after the hydrothermal treatment was washed with THE and water, and filtered, and the solid content was separated by filtration and dried to obtain a solid residue (second solid residue). The filtrate was separated into oil and water, and THE was removed from the oil phase to obtain a solvent-soluble content. The aqueous phase was used as it was as a water-soluble content. The solvent-soluble content and the water-soluble content were referred to a second liquid substance.

In the production ratio of each component in the second step, the gas component was 8%, the water-soluble content of the second liquid substance was 66%, the solvent-soluble content of the second liquid substance was 15%, and the second solid residue was 11% in terms of carbon yield where kelp seaweed as the raw material was 100%. The O/C ratio and the H/C ratio of the second solid residue were 0.8 and 0.6, respectively. The O/C ratio and the H/C ratio of the solvent-soluble content of the second liquid substance were 0.1 and 1.0, respectively. In the total of the respective components obtained in the first step and the second step, the gas component was 3%, the water-soluble content of the liquid substance was 27%, the solvent-soluble content of the liquid substance was 11%, and the solid residue was 59% in terms of carbon yield where kelp seaweed as the raw material was 100%.

### (Third Step)

The first solid residue and the second solid residue (10 g in total) were subjected to a high heat treatment (carbonization treatment) in an electric furnace under the conditions of 1000° C. and 1 hour in the air atmosphere to obtain 3.2 g of carbide. The O/C ratio and the H/C ratio of the carbide obtained in the third step were 0.03 and 0.07, respectively.

### Example 2

### (First Step)

A 9 mL SUS ½ inch reaction tube was charged with 1 g of pulverized dried kelp seaweed (Japanese kelp) and 7 ml of water, and heated in a salt bath at 200° C. for 30 minutes, and then, the reaction tube was taken out and quenched in water.

The gas component after hydrothermal treatment was collected by connecting a sampling bag to a valve previously attached to the reaction tube. A reaction mixture was washed with THE and water, and filtered, and the solid content was separated by filtration and dried to obtain the first solid residue. The filtrate was separated into oil and water, and THE was removed from the oil phase to obtain a first oil phase. The solid content of the aqueous phase was adjusted to 16 wt. & to form the first aqueous phase.

In the production ratio of each component in the first step, the gas component was 0%, the water-soluble content was 50%, the solvent-soluble content was 5%, and the solid residue was 45% in terms of carbon yield where kelp seaweed as the raw material was 100%.

### (Second Step)

4.5 g of the first aqueous phase obtained in the first step was placed in a 9 mL SUS ½ inch reaction tube, heated in a salt bath at 320° C. for 5 minutes, and then, the reaction tube was taken out and quenched in water.

The gas component, the second solid residue, the second oil phase, and the second aqueous phase after the hydrothermal treatment were recovered in the same manner as in the first step. The solid content of the aqueous phase was adjusted to 16 wt. % to form the second aqueous phase.

In the production ratio of each component in the second step, the gas component was 11%, the water-soluble content was 49%, the solvent-soluble content was 34%, and the solid residue was 6% in terms of carbon yield where carbon in the water-soluble content obtained in the first step, which was the raw material, was 100%.

### (Second Step (Recycle))

The first aqueous phase of the first step separately performed and the second aqueous phase of the second step were mixed so as to have a weight ratio of 1/1 in terms of carbon content, 4.5 g of the mixture was placed in a 9 ml SUS ½ inch reaction tube, and heated in a salt bath at 320° C. for 20 minutes, and then the reaction tube was taken out and quenched in water.

The gas component, the solid residue, the second oil phase, and the second aqueous phase after the hydrothermal treatment were recovered in the same manner as in the first step. The solid content of the aqueous phase repeatedly used was adjusted to 16 wt. % to form the second aqueous phase.

In the production ratio of each component in the second step (recycle), the gas component was 11%, the water-soluble content was 51%, the solvent-soluble content was 35%, and the solid residue was 6% in terms of carbon yield where carbon in the water-soluble content of the first step and the water-soluble content of the second step, which were the raw materials, was 100%.

In the second step (recycle), the second aqueous phase of the second step, which is always produced, is added to the first aqueous phase of the first step to cause a reaction; therefore, by repeating the second step (recycle), the percentage of the water-soluble content in the whole product is reduced, and theoretically, in terms of carbon yield where kelp seaweed as the raw material is 100%, the gas component would be 11%, the water-soluble content would be 0%, the solvent-soluble content would be 38%, and the solid residue would be 51%.

As a result, it is possible to increase the solvent-soluble content that can be used for tar, pitch, fuel oil, and the like while maintaining an amount of solid residue that can be converted to carbon by post-firing without generating a water-soluble content that is difficult to use.

### Comparative Example 1

A 9 mL SUS ½ inch reaction tube was charged with 1 g of pulverized dried kelp seaweed (Japanese kelp) and 7 ml of water, and heated in a salt bath at 320° C. for 5 minutes, and then, the reaction tube was taken out and quenched in water.

The gas component, the solid residue, the oil phase, and the aqueous phase after the hydrothermal treatment were recovered in the same manner as in the first step of Example 2.

In the production ratio of each component, the gas component was 25%, the water-soluble content was 15%, the solvent-soluble content was 46%, and the solid residue was 14% in terms of carbon yield where kelp seaweed as the raw material was 100%. Although the solvent-soluble content is large, the amount of solid residue is small, and the yield of carbon obtained by post-firing the solid residue is poor.

Example 3

### (First Step)

12 g of dried kelp seaweed (Japanese kelp) was pulverized, mixed with water at a weight ratio of kelp seaweed/water=¼, and subjected to hydrothermal treatment in a salt bath at 250° C. for 5 minutes in a 75 mL SUS pressure-resistant container (autoclave). At this time, the pressure in the autoclave was controlled to 3 MPa or less.

A reaction mixture after the hydrothermal treatment was washed with THE and water, and filtered, and the solid content was separated by filtration and dried to obtain a solid residue (first solid residue). The filtrate was separated into oil and water, and THE was removed from the oil phase to obtain a solvent-soluble content. The aqueous phase was used as it was as a water-soluble content. The solvent-soluble content and the water-soluble content were both referred to the first liquid substance.

In the production ratio of each component in the first step, the gas component was 8%, the water-soluble content of the first liquid substance was 42%, the solvent-soluble content of the first liquid substance was 7%, and the first solid residue was 43% in terms of carbon yield where kelp seaweed as the raw material was 100%. The O/C ratio and the H/C ratio of the first solid residue were 0.3 and 1.1, respectively. The O/C ratio and the H/C ratio of the solvent-soluble content of the first liquid substance were 0.13 and 1.49, respectively.

### (Second Step)

The water-soluble content (0.15 g as carbon) obtained in the first step was heat-treated at 350° C. for 5 minutes in a 10 mL SUS pressure-resistant container (autoclave). At this time, the pressure in the autoclave was controlled to 20 MPa or less.

A reaction mixture after the hydrothermal treatment was washed with THE and water, and filtered, and the solid content was separated by filtration and dried to obtain a solid residue (second solid residue). The filtrate was separated into oil and water, and THE was removed from the oil phase to obtain a solvent-soluble content. The aqueous phase was used as it was as a water-soluble content. The solvent-soluble content and the water-soluble content were both referred to the second liquid substance.

In the production ratio of each component in the second step, the gas component was 8%, the water-soluble content of the second liquid substance was 66%, the solvent-soluble content of the second liquid substance was 15%, and the second solid residue was 11% in terms of carbon yield where kelp seaweed as the raw material was 100%. The O/C ratio and the H/C ratio of the second solid residue were 0.8 and 0.6, respectively. The O/C ratio and the H/C ratio of the solvent-soluble content of the second liquid substance were 0.1 and 1.0, respectively. In the total of the respective components obtained in the first step and the second step, the gas component was 12%, the water-soluble content of the liquid substance was 28%, the solvent-soluble content of the liquid substance was 13%, and the solid residue was 47% in terms of carbon yield where kelp seaweed as the raw material was 100%.

### Comparative Example 2

A 9 mL SUS ½ inch reaction tube was charged with 1 g of pulverized dried kelp seaweed (Japanese kelp) and 7 ml of water, and heated in a salt bath at 250° C. for 5 minutes, and then, the reaction tube was taken out and quenched in water.

The gas component, the solid residue, the oil phase, and the aqueous phase after the hydrothermal treatment were recovered in the same manner as in the first step of Example 2.

In the production ratio of each component, the gas component was 8%, the water-soluble content was 42%, the solvent-soluble content was 7%, and the solid residue was 43% in terms of carbon yield where kelp seaweed as the raw material was 100%. The O/C ratio and the H/C ratio of the first solid residue were 0.3 and 1.1, respectively. The O/C ratio and the H/C ratio of the solvent-soluble content of the first liquid substance were 0.13 and 1.49, respectively.

### Comparative Example 3

A 9 mL SUS ½ inch reaction tube was charged with 1 g of pulverized dried kelp seaweed (Japanese kelp) and 7 ml of water, and heated in a salt bath at 350° C. for 5 minutes, and then, the reaction tube was taken out and quenched in water.

The gas component, the solid residue, the oil phase, and the aqueous phase after the hydrothermal treatment were recovered in the same manner as in the first step of Example 2.

In the production ratio of each component, the gas component was 25%, the water-soluble content was 15%, the solvent-soluble content was 46%, and the solid residue was 14% in terms of carbon yield where kelp seaweed as the raw material was 100%. The O/C ratio and the H/C ratio of the first solid residue were 1.1 and 0.6, respectively. The O/C ratio and the H/C ratio of the solvent-soluble content of the first liquid substance were 0.1 and 1.0, respectively.

### Comparative Example 4

### (First Step)

12 g of dried kelp seaweed (Japanese kelp) was pulverized, mixed with water at a weight ratio of kelp seaweed/water=¼, and subjected to hydrothermal treatment in a salt bath at 270° C. for 5 minutes in a 75 mL SUS pressure-resistant container (autoclave). At this time, the pressure in the autoclave was controlled to 3 MPa or less.

A reaction mixture after the hydrothermal treatment was washed with THF and water, and filtered, and the solid content was separated by filtration and dried to obtain a solid residue (first solid residue). The filtrate was separated into oil and water, and THE was removed from the oil phase to obtain a solvent-soluble content. The aqueous phase was used as it was as a water-soluble content. The solvent-soluble content and the water-soluble content were both referred to the first liquid substance.

In the production ratio of each component in the first step, the gas component was 13%, the water-soluble content of the first liquid substance was 38%, the solvent-soluble content of the first liquid substance was 9%, and the first solid residue was 40% in terms of carbon yield where kelp seaweed as the raw material was 100%. The O/C ratio and the H/C ratio of the first solid residue were 0.22 and 0.9, respectively. The O/C ratio and the H/C ratio of the solvent-soluble content of the first liquid substance were 0.12 and 1.4, respectively.

### (Second Step)

The water-soluble content (0.15 g as carbon) obtained in the first step was heat-treated at 300° C. for 5 minutes in a 10 ML SUS pressure-resistant container (autoclave). At this time, the pressure in the autoclave was controlled to 20 MPa or less.

A reaction mixture after the hydrothermal treatment was washed with THE and water, and filtered, and the solid content was separated by filtration and dried to obtain a solid residue (second solid residue). The filtrate was separated into oil and water, and THE was removed from the oil phase to obtain a solvent-soluble content. The aqueous phase was used as it was as a water-soluble content. The solvent-soluble content and the water-soluble content were both referred to the second liquid substance.

In the production ratio of each component in the second step, the gas component was 2%, the water-soluble content of the second liquid substance was 89%, the solvent-soluble content of the second liquid substance was 4%, and the second solid residue was 5% in terms of carbon yield where kelp seaweed as the raw material was 100%. The O/C ratio and the H/C ratio of the second solid residue were 0.7 and 0.65, respectively. The O/C ratio and the H/C ratio of the solvent-soluble content of the second liquid substance were 0.11 and 1.3, respectively. In the total of the respective components obtained in the first step and the second step, the gas component was 14%, the water-soluble content of the liquid substance was 35%, the solvent-soluble content of the liquid substance was 10%, and the solid residue was 41% in terms of carbon yield where kelp seaweed as the raw material was 100%.

### Comparative Example 5

### (First Step)

12 g of dried kelp seaweed (Japanese kelp) was pulverized, mixed with water at a weight ratio of kelp seaweed/water=¼, and subjected to hydrothermal treatment at 180° C. for 10 minutes in a 75 mL SUS pressure-resistant container (autoclave). At this time, the pressure in the autoclave was controlled to 3 MPa or less.

A reaction mixture after the hydrothermal treatment was washed with THF and water, and filtered, and the solid content was separated by filtration and dried to obtain a solid residue (first solid residue). The filtrate was separated into oil and water, and THE was removed from the oil phase to obtain a solvent-soluble content. The aqueous phase was used as it was as a water-soluble content. The solvent-soluble content and the water-soluble content were both referred to the first liquid substance.

In the production ratio of each component in the first step, the gas component was 0%, the water-soluble content of the first liquid substance was 36%, the solvent-soluble content of the first liquid substance was 4%, and the first solid residue was 60% in terms of carbon yield where kelp seaweed as the raw material was 100%. The O/C ratio and the H/C ratio of the first solid residue were 0.48 and 1.3, respectively. The O/C ratio and the H/C ratio of the solvent-soluble content of the first liquid substance were 0.15 and 1.72, respectively.

### (Second Step)

The water-soluble content (0.15 g as carbon) obtained in the first step was heat-treated at 300° C. for 5 minutes in a 10 mL SUS pressure-resistant container (autoclave). At this time, the pressure in the autoclave was controlled to 20 MPa or less.

A reaction mixture after the hydrothermal treatment was washed with THE and water, and filtered, and the solid content was separated by filtration and dried to obtain a solid residue (second solid residue). The filtrate was separated into oil and water, and THE was removed from the oil phase to obtain a solvent-soluble content. The aqueous phase was used as it was as a water-soluble content. The solvent-soluble content and the water-soluble content were both referred to the second liquid substance.

In the production ratio of each component in the second step, the gas component was 2.5%, the water-soluble content of the second liquid substance was 89%, the solvent-soluble content of the second liquid substance was 2.5%, and the second solid residue was 6% in terms of carbon yield where kelp seaweed as the raw material was 100%. The O/C ratio and the H/C ratio of the second solid residue were 0.3 and 1.1, respectively. The O/C ratio and the H/C ratio of the solvent-soluble content of the second liquid substance were 0.12 and 1.5, respectively. In the total of the respective components obtained in the first step and the second step, the gas component was 1%, the water-soluble content of the liquid substance was 33%, the solvent-soluble content of the liquid substance was 4%, and the solid residue was 62% in terms of carbon yield where kelp seaweed as the raw material was 100%.

### Industrial Applicability

According to the present invention, there is provided a process for efficiently producing useful liquid hydrocarbon, preferably liquid hydrocarbon and carbide, utilizing seaweeds which is biomass.

1. A method for industrially producing resources from seaweeds, the method comprising:
a first step of performing a low-temperature hydrothermal treatment at from 120° C. to 250° C. using seaweeds and then performing solid-liquid separation to obtain a first liquid substance and a first solid residue having an O/C ratio of from 0.3 to 0.8; and
a second step of performing a high-temperature hydrothermal treatment at from 250° C. to 370° C. using a first aqueous phase obtained from the first liquid substance obtained in the first step and then performing solid-liquid separation to obtain a second liquid substance including a heavy oil-containing liquid substance and a second solid residue.

2. The method according to claim 1, further comprising a third step of performing a high heat treatment at from 400° C. to 1000° C. using the first solid residue and/or the second solid residue to obtain carbide.

3. The method according to claim 1, wherein the seaweeds are at least one selected from the group consisting of green algae, brown algae, and red algae.

4. The method according to claim 2, wherein the carbide obtained in the third step is activated carbon.

5. The method according to claim 1, wherein the second liquid substance obtained in the second step is separated into oil and water to obtain a heavy oil-containing liquid substance comprising light oil, heavy oil, pitch, tar, or a mixture of two or more kinds thereof and an aqueous phase.

6. The method according to claim 5, wherein the aqueous phase obtained in the second step is subjected to a fermentation step of fermenting the aqueous phase with microorganisms to obtain a combustible gas containing methane.

7. The method according to claim 1, wherein at least a part of the second aqueous phase obtained from the second liquid substance obtained in the second step is circulated to a step of performing the high-temperature hydrothermal treatment together with the first aqueous phase obtained from the first liquid substance obtained in the first step.

8. The method according to claim 7, further comprising a purification step of distilling a first oil phase obtained from the first liquid substance obtained in the first step and a second oil phase obtained from the second liquid substance obtained in the second step to obtain light oil and heavy oil.
